(19) United States
(12) Patent Application Publication
(10) Pub. No.: US 2003/0113286 A1
(43) Pub. Date: Jun. 19, 2003

(54) Hair colouring and conditioning compositions

(75) Inventors: Nicholas William Geary, Blue Ash, OH (US); Dieter Hans Josef Langsch, Richmond (GB); Simon Paul Godfrey, Egham (GB)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company, Cincinnati, OH (US)

(21) Appl. No.: 10/264,500

(22) Filed: Oct. 4, 2002

Related U.S. Application Data
(63) Continuation of application No. PCT/US01/10486, filed on Mar. 30, 2001.

(30) Foreign Application Priority Data
Aug. 11, 2000 (GB) 0019834.1
Apr. 7, 2000 (GB) 0008677.7

(51) Int. Cl. A61K 7/06; A61K 7/11; A61K 7/13

(52) U.S. Cl. 424/70.12; 8/406

(57) ABSTRACT

A hair colouring and conditioning composition comprising:

(a) a hair colouring agent; and

(b) a hair conditioning agent;

wherein the composition provides an Average Combing Index Value of greater than 1.2 as measured by the Combing Technical Test Method.

The products can provide excellent hair colouring together with excellent conditioning, reduced hair damage, brittleness and dryness, and is convenient and easy to use.

### CROSS REFERENCE TO RELATED APPLICATION

The application is a continuation of International application PCT/US01/10486 (Case CM2310FM2) filed on Mar. 30, 2001.

### TECHNICAL FIELD

This invention relates to hair colouring compositions and methods for colouring hair, and more especially to hair colouring compositions, which provide both colouring and conditioning to the hair from a single product.

### BACKGROUND OF THE INVENTION

The desire to alter the color of human hair is not a facet of modern times. Since the days of the Roman Empire the color of human hair has been routinely altered to accommodate the changes of fashion and style. However the attainment of precise initial colors, which are retained by the hair for a desirable period has remained a more elusive goal. The difficulties in the development of hair coloring compositions which can deliver precise long-lasting colors are in part due to the inherent structure of the hair itself and in part due to the necessary conditions of effective hair coloration processes. Common difficulties with conventional hair coloring compositions include color fade, wash fade, irregular dye uptake, hair damage and brittleness, skin irritation, odor and skin staining.

Over the years significant effort has been directed towards the elimination of many of the problems associated with the dyeing of human hair. Various approaches to hair dyeing have been developed, these include, direct action dyes, natural dyes, metallic dyes and oxidative dyes.

However there is still a need for a significant improvement in a couple of key areas. In particular, it is desired to provide a hair colouring composition which delivers excellent colour to the hair but which, at the same time, reduces the damaging effects commonly associated with hair dyes, especially oxidative hair dyes. Damaging effects include tangling, brittleness, dryness and hair breakage. In addition, it is desired to provide a hair colouring composition, which is convenient and quick to use and which can be used as part of the conventional hair cleansing and conditioning regime used by consumers in the bath or shower. Many currently available hair colouring compositions, for example, those based on oxidative dye technology, suffer from the disadvantage that they need to be left on the hair for a relatively long period of time, on average around 30 minutes, in order to allow enough time for a colour to develop on the hair. In addition, many such compositions are inconvenient to use. For example, in the case of conventional hair colouring compositions using oxidative dye technology the hair is treated with a mixture of oxidative hair colouring agents and an oxidising agent. Hydrogen peroxide is the most commonly used oxidising agent. Usually, the product is sold as a kit containing separately packaged hair dye components and oxidising components. The consumer is then instructed to mix the two components together prior to application on the head. This is time consuming and messy for the consumer.

It has now surprisingly been found that the hair colouring composition of the present invention containing a hair colouring agent and a hair conditioning agent, wherein the composition has a certain level of conditioning, overcomes the problems of conventional hair colouring compositions based on oxidative dye technology, in particular in relation to providing excellent conditioning, reduced hair damage, tangling, brittleness, dryness and messiness and increased convenience and ease of use. Indeed, it is an object of the present invention to provide a hair colouring composition which can be used as part of the conventional cleansing and conditioning routine used by consumers in the bath and shower.

All percentages are by weight of the final compositions in the form intended to be used unless specified otherwise.

### SUMMARY OF THE INVENTION

The subject of the present invention is a hair colouring and conditioning composition suitable for the treatment of human or animal hair.

According to one aspect of the present invention, there is provided a hair colouring and conditioning composition comprising:

(a) at least one conditioning agent; and

(b) at least one hair colouring agent;

wherein the composition has an Average Combing Index Value of greater than 1.2 as measured by the Combing Technical Test Method as defined herein below.

It is to be understood that the percentage weights of the composition components herein are expressed in terms of the total composition, and refers to the composition in the form of intended use, i.e. the final composition which is intended for application to the hair.

According to a further aspect of the present invention, there is provided a method for colouring and conditioning hair wherein a hair colouring and conditioning composition is applied directly to the hair and subsequently rinsed off, wherein the hair colouring and conditioning composition comprises:

(a) at least one conditioning agent;

(b) at least one hair colouring agent;

wherein the composition has a Combing Index Value of greater than 1.2 as measured by the Combing Technical Test Method as defined herein.

According to yet another aspect of the present invention, there is provided a hair colouring and conditioning kit comprising at least two components, a first component comprising an oxidising agent and a second component comprising a hair colouring agent, provided at least one of the components comprises a conditioning agent, wherein the two components are mixed together immediately before application to the hair from a hair colouring and conditioning composition.

In some embodiments the kit may additionally comprise an initial colouring composition suitable for providing an initial colour to hair before the use of the hair colour altering composition. The consumer applies colour initially using the initial colouring composition and maintains the colour over the subsequent weeks by the use of the hair colour altering composition. Generally the initial colouring composition and hair colour altering composition are of substantially the same shade.

### DETAILED DESCRIPTION OF THE INVENTION

The hair colouring and conditioning composition herein comprises at least one conditioning agent and at least one hair colouring agent. In addition, the hair colouring and conditioning composition provides an Average Combing Index Value of greater than 1.2 as measured by the Combing Technical Test Method described herein below.

As used herein the term ‘hair’ to be treated may be ‘living’ i.e. on a living body or may be ‘non-living’ i.e. in a wig, hairpiece or other aggregation of non-living fibres, such as those used in textiles and fabrics. Mammalian, preferably human hair is preferred. However wool, fur, and other melanin containing fibres are suitable substrates for the compositions according to the present invention.

As used herein the term ‘hair colouring and conditioning composition’ is used in the broad sense in that it is intended to encompass compositions containing the combinations herein of at least one conditioning agent and at least one hair colouring agent wherein the composition has an Average Combing Index Value of greater than 1.2 as measured by the Combing Technical Test Method as defined herein. Moreover, it is also intended to include complex compositions which contain other components which may or may not be active ingredients. Thus, the term ‘hair colouring and conditioning composition’ is intended to apply to compositions which contain, in addition to a mixture of at least one conditioning agent and at least one hair colouring agent, such things as, by way of example, but not limited to oxidising agents, oxidising aids, sequestrants, stabilisers, thickeners, buffers, carriers, surfactants, solvents, antioxidants, polymers, UV filters and additional conditioning agents.

Conditioning Agent

The conditioning agent herein can be any conditioning agent suitable for use in conditioning hair provided that the hair colouring and conditioning composition provides an Average Combing Index Value of greater than 1.2 as measured by the Combing Technical Test Method.

The hair colouring and conditioning compositions herein comprise one or more conditioning agents preferably in the level of from about 0.1% to about 25%, preferably from about 1% to about 20%, more preferably from about 5% to about 20% and especially from about 5% to about 15%, by weight of the hair colouring and conditioning composition. Suitable conditioning agents for use herein include, but are not limited to, cationic surfactants, cationic polymers, insoluble silicones, non-volatile hydrocarbons, saturated C14-C22 straight chain fatty alcohols, non-volatile hydrocarbon esters, and mixtures thereof . Other suitable conditioning agents are disclosed in WO95/20939, which is incorporated herein by reference.

Preferred conditioning agents for use herein include cationic polymers, non-volatile silicones and saturated C14-C22 straight chain fatty alcohols and mixtures thereof. Especially preferred for use herein is a mixture of cationic polymer, non-volatile silicone and C14-C22 straight chain fatty alcohols.

When present, the non-volatile silicones are present at a level of from about 0.1 to 10%, preferably from about 0.1% to about 5%, more preferably from about 1% to about 3% by weight of composition.

When present, the cationic polymers are present at a level of from about 0.1 to 10%, preferably from about 0.1% to about 5%, more preferably from about 1% to about 3% by weight of composition.

When present, the fatty alcohols are present at a level of from about from about 0.1% to about 20%, preferably from about 1% to about 15% and more preferably from about 3% to about 10% by weight of composition.

Hair Colouring Agents

The hair colouring agent herein can be any colouring agent suitable for use in colouring hair. Suitable hair colouring agents include, but are not limited to, oxidative dyes, photographic dyes, acid dyes, neutral dyes, reactive dyes, cationic dyes, VAT dyes, and mixtures thereof. Particularly preferred for use herein are oxidative dyes.

The hair coloring compositions of the present invention preferably include an oxidative or non-oxidative hair coloring agent. Such hair coloring agents are used in combination with the conditioning agent to formulate permanent, demi-permanent or semi-permanent hair dye compositions having superior hair conditioning properties.

Permanent hair dye compositions as defined herein are compositions which once applied to the hair are substantially resistant to wash-out. Demi-permanent hair dye compositions as defined herein are compositions which are substantially removed from the hair after up to 24 washes. Semi-permanent hair dye compositions as defined herein are compositions which once applied to the hair are substantially removed from the hair after up to 10 washes. Temporary hair dye compositions as defined herein are compositions which once applied to the hair are substantially removed from the hair after up to 2 washes. These different types of hair coloring compositions can be formulated via the specific combination of oxidant and/or dyes at different levels and ratios. Wash out as defined herein is the process by which hair color is removed from the hair over time during normal hair cleansing regimen. Washfastness as defined herein, means, the resistance of the dyed hair to wash out.

Oxidative Hair Coloring Agents

A preferred hair coloring agent herein is an oxidative hair coloring agent. The concentration of each oxidative hair coloring agent in the coloring compositions according to the present invention is preferably from about 0.001% to about 3% by weight, more preferably from about 0.01% to about 2% by weight.

The total combined level of oxidative hair coloring agents in the compositions according to the present invention is from about 0.001% to about 5%, preferably from about 0.01% to about 4%, more preferably from about 0.1% to about 3%, most preferably from about 0.1% to about 1% by weight.

Any oxidative hair coloring agent can be used in the compositions according to the present invention. Typically, but without intending to be limited thereby, oxidative hair coloring agents, consist essentially of at least two components, which are collectively referred to as dye forming intermediates (or precursors). Dye forming intermediates can react in the presence of a suitable oxidant to form a colored molecule.

The dye forming intermediates used in oxidative hair colorants include: aromatic diamines, aminophenols, various heterocycles, phenols, napthols and their various derivatives. These dye forming intermediates can be broadly classified as; primary intermediates and secondary intermediates. Primary intermediates, which are also known as oxidative dye precursors, are chemical compounds which become activated upon oxidation and can then react with each other and/or with couplers to form colored dye complexes. The secondary intermediates, also known as color modifiers or couplers, are generally colorless molecules which can form colors in the presence of activated precursors/primary intermediates, and are used with other intermediates to generate specific color effects or to stabilise the color.

Primary intermediates suitable for use in the compositions and processes herein include: aromatic diamines, polyhydric phenols, amino phenols and derivatives of these aromatic compounds (e.g., N-substituted derivatives of the amines, and ethers of the phenols). Such primary intermediates are generally colorless molecules prior to oxidation.

While not wishing to be bound by any particular theory it is proposed herein that the process by which color is generated from these primary intermediates and secondary coupler compounds generally includes a stepwise sequence whereby the primary intermediate can become activated (by oxidation), and then enjoins with a coupler to give a dimeric, conjugated colored species, which in turn can enjoin with another ‘activated’ primary intermediate to produce a trimeric conjugated colored molecule.

Oxidative Dye Precursors

In general terms, oxidative dye primary intermediates include those monomeric materials which, on oxidation, form oligomers or polymers having extended conjugated systems of electrons in their molecular structure. Because of the new electronic structure, the resultant oligomers and polymers exhibit a shift in their electronic spectra to the visible range and appear colored. For example, oxidative primary intermediates capable of forming colored polymers include materials such as aniline, which has a single functional group and which, on oxidation, forms a series of conjugated imines and quinoid dimers, trimers, etc. ranging in color from green to black. Compounds such as p-phenylenediamine, which has two functional groups, are capable of oxidative polymerization to yield higher molecular weight colored materials having extended conjugated electron systems. Oxidative dyes known in the art can be used in the compositions according to the present invention. A representative list of primary intermediates and secondary couplers suitable for use herein is found in Sagarin, “Cosmetic Science and Technology”, Interscience, Special Ed. Vol. 2 pages 308 to 310. It is to be understood that the primary intermediates detailed below are only by way of example and are not intended to limit the compositions and processes herein. The typical aromatic diamines, polyhydric phenols, amino phenols, and derivatives thereof, described above as primary intermediates can also have additional substituents on the aromatic ring, e.g. halogen, aldehyde, carboxylic acid, nitro, sulfonic acid and substituted and unsubstituted hydrocarbon groups, as well as additional substituents on the amino nitrogen and on the phenolic oxygen, e.g. substituted and unsubstituted alkyl and aryl groups.

Examples of suitable aromatic diamines, amino phenols, polyhydric phenols and derivatives thereof, respectively, are compounds having the general formulas (I), (II) and (III) below:

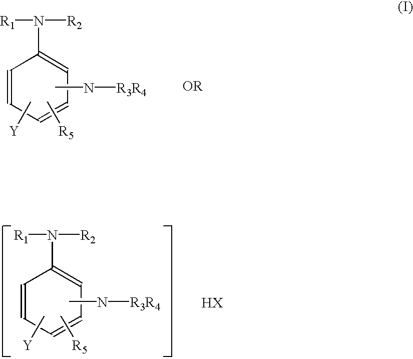

wherein Y is hydrogen, halogen, (e.g. fluorine, chlorine, bromine or iodine), nitro, amino, hydroxyl,

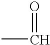

—COOM or —SO₃M (where M is hydrogen or an alkali or alkaline earth metal, ammonium, or substituted ammonium wherein one or more hydrogens on the ammonium ion is replaced with a 1 to 3 carbon atom alkyl or hydroxyalkyl radical), wherein R₁, R₂, R₃ and R₄ are the same or different from each other and are selected from the group consisting of hydrogen, C₁ to C₄ alkyl or alkenyl and C₆ to C₉ aryl, alkaryl or aralkyl, and R₅ is hydrogen, C₁ to C₄ unsubstituted or substituted alkyl or alkenyl wherein the substituents are selected from those designated as Y, above, or C₆ to C₉ unsubstituted or substituted aryl, alkaryl or aralkyl wherein the substituents are selected from those defined as Y, above. Since the precursors of formula (I) are amines, they can be used herein in the form of peroxide-compatible salts, as noted, wherein X represents peroxide-compatible anions of the type herein before detailed. The general formula of the salt indicated is to be understood to encompass those salts having mono-, di-, and tri-negative anions.

Specific examples of formula (I) compounds are: o-phenylenediamine, m-phenylenediamine, p-phenylenediamine, 2-chloro-p-phenylenediamine, 2-iodo-p-phenylenediamine, 4-nitro-o-phenylenediamine, 2-nitro-p-phenylenediamine, 1,3,5-triaminobenzene, 2-hydroxy-p-phenylenediamine, 2,4-diaminobenzoic acid, sodium 2,4-diaminobenzoate, calcium di-2,4-diaminobenzoate, ammonium 2,4-diaminobenzoate, trimethylammonium 2,4-, diaminobenzoate, tri-(2-hydroxyethyl)ammonium 2,4-diaminobenzoate, 2,4-diaminobenzaldehyde carbonate, 2,4-diaminobenzensulfonic acid, potassium 2,4-diaminobenzenesulfonate, N,N-diisopropyl-p-, phenylenediamine bicarbonate, N,N-dimethyl-p-phenylenediamine, N-ethyl-N′-(2-propenyl)-p-phenylenediamine, N-phenyl-p-phenylenediamine, N-phenyl-N-benzyl-p-phenylenediamine, N-ethyl-N′-(3-ethylphenyl)-p-phenylenediamine, 2,4-toluenediamine, 2-ethyl-p-phenylenediamine, 2-(2-bromoethyl)-p-phenylenediamine, 2-phenyl-p-phenylenediamine laurate, 4-(2,5-diaminophenyl)benzaldlehyde, 2-benzyl-p-phenylenediamine acetate, 2-(4-nitrobenzyl)-p-phenylenediamine, 2-(4-methylphenyl)-p-phenylenediamine, 2-(2,5-diaminophenyl)-5-methylbenzoic acid, methoxyparaphenylenediamine, dimethyl-p-phenylenediamine, 2,5-dimethylpara-phenylenediamine, 2-methyl-5-methoxy-para-phenylenediamine, 2,6-methyl-5-methoxy-para-phenylenediamine, 3-methyl-4-amino-N,N-diethylaniline, N,N-bis(□-hydroxyethyl)-para-phenylenediamine, 3-methyl-4-amino-N,N-bis(□-hydroxyethyl)aniline, 3-chloro-4-amino-N,N-bis( □-hydroxyethyl)aniline, 4-amino-N-ethyl-N-(carbamethyl)aniline, 3-methyl-4-amino-N-ethyl-N-(carbamethyl)aniline, 4-amino-N-ethyl-(□-piperidonoethyl)aniline, 3-methyl-4-amino-N-ethyl-(□-piperidonoethyl)aniline, 4-amino-N-ethyl-N-(□-morpholinoethyl)aniline, 3-methyl-4-amino-N-ethyl-N-(□-morpholinoethyl)aniline, 4-amino-N-ethyl-N-(□-acetylaminoethyl)aniline, 4-amino-N-(□-methoxyethyl) aniline, 3-methyl-4-amino-N-ethyl-N-(□-acetylaminoethyl) aniline, 4-amino-N-ethyl-N-(□-mesylaminoethyl) aniline, 3-methyl4-amino-N-ethyl-N-(□-mesylaminoethyl) aniline, 4-amino-N-ethyl-N-(□-sulphoethyl) aniline, 3-methyl-4-amino-N-ethyl-N-(□-sulphoethyl) aniline, N-(4-aminophenyl)morpholine, N-(4-aminophenyl)piperidine, 2,3-dimethyl-p-phenylenediamine, isopropyl-p-phenylenediamine, N,N-bis-(2-hydroxyethyl)-p-phenylenediamine sulphate.

Compounds having the general structure (II) are as follows:

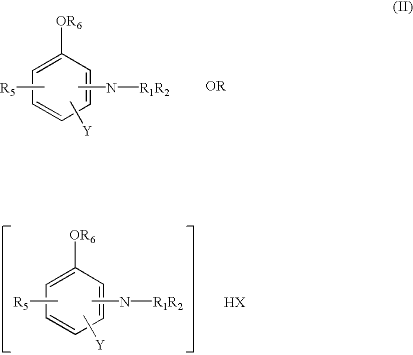

where X and Y are the same as in formula (I), R₁ and R₂ can be the same or different from each other and are the same as in formula (I), R₅ is the same as in formula (I) and R₆ is hydrogen or C₁ to C₄ substituted or unsubstituted alkyl or alkenyl wherein the substituents are selected from those defined as Y in formula (I).

Specific examples of formula (II) compounds are:

o-aminophenol, m-aminophenol, p-aminophenol, 2-iodo-p-aminophenol, 2-nitro-p-aminophenol, 3,4-dihydroxyaniline, 3,4-diaminophenol, chloroacetate, 2-hydroxy-4-aminobenzoic acid, 2-hydroxy-4-aminobenzaldehyde, 3-amino-4-hydroxybenzenesulfonic acid, N,N-diisopropyl-p-aminophenol, N-methyl-N-( 1 -propenyl)-p-aminophenol, N-phenyl-N-benzyl-p-aminophenol sulphate, N-methyl-N-(3-ethylphenyl)-p-aminophenol, 2-nitro-5-ethyl-p-aminophenol, 2-nitro-5-(2-bromoethyl)-p-aminophenol, (2-hydroxy-5-aminophenyl)acetaldehyde, 2-methyl-p-aminophenol, (2-hydroxy-5-aminophenyl)acetic acid, 3-(2-hydroxy-5-aminophenyl)-1-propene, 3-(2-hydroxy-5-aminophenyl)-2-chloro-1-propene, 2-phenyl-p-aminophenol palmitate, 2-(4-nitrophenyl)-p-aminophenol, 2-benzyl-p-aminophenol, 2-(4-chlorobenzyl-p-aminophenol perchlorate, 2-(4-methylphenyl)-p-aminophenol, 2-(2-amino-4-methylphenyl)-p-aminophenol, p-methoxyaniline, 2-bromoethyl-4-aminophenyl ether phosphate, 2-nitroethyl-4-aminophenyl ether bromide, 2-aminoethyl4-aminophenyl ether , 2-hydroxyethyl4-aminophenyl ether, (4-aminophenoxy)acetaldehyde, (4-aminophenoxy)acetic acid, (4-aminophenoxy)methanesulfonic acid, 1-propenyl4-aminophenyl ether isobutyrate, (2-chloro)-1-propenyl-4-aminophenyl ether, (2-nitro)-1-propenyl4-aminophenyl ether, (2-amino)-propenyl4-aminophenyl ether, (2-hydroxy)-1-propenyl-4-aminophenyl ether, N-methyl-p-aminophenol, 3-methyl-4-aminophenol, 2-chloro-4-aminophenol, 3-chloro-4-aminophenol, 2,6-dimethyl-4-aminophenol, 3,5-dimethyl-4-aminophenol, 2,3-dimethyl-4-aminophenol, 2,5-dimethyl-4-aminophenol, 2-hydroxymethyl-4-aminophenol, 3-hydroxymethyl-4-aminophenol, 2,6-dichloro-4-aminophenol, 2,6-dibromo-4-aminophenol and 2-bromo-4-aminophenol.

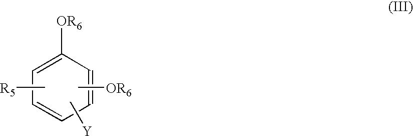

Specific examples of formula (III) compounds are:

o-hydroxyphenol (catechol), m-hydroxyphenol (resorcinol), p-hydroxyphenol (hydroquinone), 4-methoxyphenol, 2-methoxyphenol, 4-(2-chloroethoxy) phenol, 4-(2-propenoxy) phenol, 4-(3-chloro-2-propenoxy) phenol, 2-chloro-4-hydroxyphenol (2-chlorohydroquinone), 2-nitro-4-hydroxyphenol(2-nitrohydroquinone), 2-amino-4-hydroxyphenol, 1,2,3-trihydroxybenzene (pyrogallol), 2,4-dihydroxybenzaldehyde, 3,4-dihydoxybenzoic acid, 2,4-dihydroxybenzenesulfonic acid, 3-ethyl-4-hydroxyphenol, 3-(2-nitroethyl)-4-hydroxyphenol, 3-(2-propenyl)4-hydroxyphenol, 3-(3-chloro-2-propenyl)-4-hydroxyphenol, 2-phenyl-4-hydroxyphenol, 2-(4-chlorophenyl)-4-hydroxyphenol, 2-benzyl-4-hydroxyphenol, 2-(2-nitrophenyl)4-hydroxyphenol, 2-(2-methylphenyl)4-hydroxyphenol, 2-(2-methyl-4-chlorophenyl)4-hydroxyphenol, 3-methoxy-4-hydroxy-benzaldehyde, 2-methoxy-4-(1-propenyl)phenol, 4-hydroxy-3-methoxycinnamic acid, 2,5-dimethoxyaniline, 2-methylresorcinol, alpha napthol and salts thereof.

Secondary coupling compounds which are suitable for inclusion in the coloring compositions and processes herein before described include certain aromatic amines and phenols and derivatives thereof which do not produce color singly, but which modify the color, shade or intensity of the colors developed by the primary oxidized dye intermediates. Certain aromatic amines and phenolic compounds, and derivatives thereof, including some aromatic diamines and polyhydric phenols of the types described by formulas (I), (Ia), (Ib), (II) and (III) above, but which are well known in the art not to be suitable primary intermediates, are suitable as couplers herein. Polyhydric alcohols are also suitable for use as couplers herein.

The aromatic amines and phenols and derivatives described above as couplers can also have additional substituents on the aromatic ring, e.g., halogen, aldehyde, carboxylic acid, nitro, sulfonyl and substituted and unsubstituted by hydrocarbon groups, as well as additional substituents on the amino nitrogen, or phenolic oxygen, e.g. substituted and unsubstituted alkyl and aryl groups. Again, peroxide-compatible salts thereof are suitable for use herein.

Examples of aromatic amines, phenols and derivatives thereof are compounds of the general formulas (IV) and (V) below:

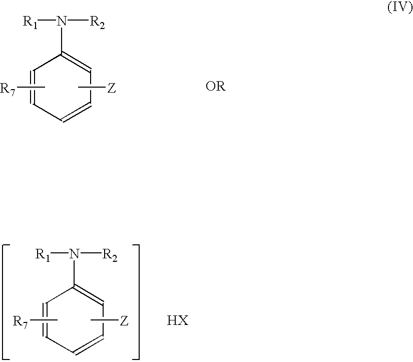

wherein Z is hydrogen, C₁ and C₃ alkyl, halogen (e.g. fluorine, chlorine, bromine or iodine) nitro,

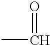

—COOM or SO₃M, (where M is hydrogen or an alkali or alkaline earth metal, ammonium or substituted ammonium wherein one or more hydrogens on the ammonium ion is replaced with a 1 to 3 carbon atom alkyl or hydroxyalkyl radical), wherein R₁ and R₂ are the same or different and are selected from the group consisting of hydrogen, C₁ to C₄ alkyl or alkenyl and C₆ to C₉ aryl, alkaryl or aralkyl and R₇ is hydrogen, C₁ to C₄ unsubstituted or substituted alkyl or alkenyl wherein the substituents are selected from those designated as Z above or C₆ to C₉ unsubstituted or substituted aryl, alkaryl or aralkyl wherein the substituents are selected from those defined as Z above and wherein X is as defined in formula (I).

Specific examples of formula (IV) compounds are:

aniline, p-chloroaniline, p-fluoroaniline, p-nitroaniline, p-aminobenzaldehyde, p-aminobenzoic acid, sodium-p-aminobenzoate, lithium-p-aminobenzoate, calcium di-p-aminobenzoate, ammonium-p-aminobenzoate, trimethylammonium-p-aminobenzoate, tri(2-hydroxyethyl)-p-aminobenzoate, p-aminobenzenesulfonic acid, potassium p-aminobenzenesulfonate, N-methylaniline, N-propyl-N-phenylaniline, N-methyl-N-2-propenylaniline, N-benzylaniline, N-(2-ethylphenyl)aniline, 4-methylaniline, 4-(2-bromoethyl)aniline, 2-(2-nitroethyl)aniline, (4-aminophenyl)acetaldehyde, (4-aminophenyl)acetic acid, 4-(2-propenyl)aniline acetate, 4-(3-bromo-2-propenyl)aniline, 4-phenylaniline chloroacetate, 4-(3-chlorophenyl)aniline, 4-benzylaniline, 4-(4-iodobenzyl)aniline, 4-(3-ethylphenyl)aniline, 4-(2-chloro-4-ethylphenyl)aniline.

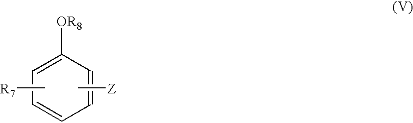

wherein Z and R₇ are defined as in formula (IV) and R₈ is hydrogen or C₁ to C₄ substituted or unsubstituted alkyl or alkenyl wherein the substituents are selected from those defined as Z in formula (IV).

Specific examples of formula (V) compounds are:

phenol, p-chlorophenol, p-nitrophenol, p-hydroxybenzaldehyde, p-hydroxybenzoic acid, p-hydroxybenzenesulfonic acid, ethylphenyl ether, 2-chloroethylphenyl ether, 2-nitroethylphenyl ether, phenoxyacetaldehyde, phenoxyacetic acid, 3-phenoxy-1-propene, 3-phenoxy-2-nitro-1-propene, 3-phenoxy-2-bromo-1-propene, 4-propylphenol, 4-(3-bromopropyl)phenol, 2-(2-nitroethyl)phenol, (4-hydroxyphenyl)acetaldehyde, (4-hydroxyphenyl)acetic acid, 4-(2-propenyl)phenol, 4-phenylphenol, 4-benzylphenol, 4-(3-fluoro-2-propenyl)phenol, 4-(4-chlorobenzyl)phenol, 4-(3-ethylphenyl)phenol, 4-(2-chloro-3-ethylphenyl)phenol, 2,5-xylenol, 2,5-diaminopyridine, 2-hydroxy-5-aminopyridine, 2-amino-3-hydroxy pyridine, tetraaminopyrimindine, 1,2,4-trihydroxybenzene, 1,2,4-trihydroxy-5-(C₁-C₆-alkyl)benzene, 1,2,3-trihydroxybenzene, 4-aminoresorcinol, 1,2-dihydroxybenzene, 2-amino-1,4-dihydroxybenzene, 2-amino-4-methoxy-phenol, 2,4-diaminophenol, 3-methoxy-1,2-dihydroxy-benzene, 1,4-dihydroxy-2-(N,N-diethylamino)benzene, 2,5-diamino-4-methoxy-1-hydroxybenzene, 4,6-dimethoxy-3-amino-1-hydroxybenzene, 2,6-dimethyl-4-[N-(p-hydroxyphenyl)amino]-1-hydroxybenzene, 1,5-diamino-2-methyl-4-[N-(p-hydroxyphenyl)amino]benzene and salts thereof.

Additional primary intermediates suitable for use herein include catechol species and in particular catechol “dopa” species which includes dopa itself as well as homologs, analogs and derivatives of DOPA. Examples of suitable cachetol species include cysteinyl dopa, alpha alkyl dopa having 1 to 4 , preferably 1 to 2 carbon atoms in the alkyl group, epinephrine and dopa alkyl esters having 1 to 6 , preferably 1 to 2 carbon atoms in the alkyl group.

In general suitable catechols are represented by formula (VI) below:

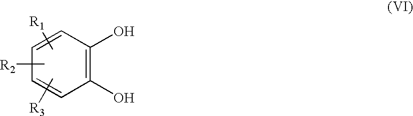

wherein R₁, R₂ and R₃, which may be the same or different, are electron donor or acceptor substutuents selected from H, lower (C₁-C₆) alkyl, OH, OR, COOR, NHCOR, CN, COOH, Halogen, NO₂, CF₃, SO₃H or NR₄R₅, with the proviso that only one of the R₁, R₂ or R₃ can be CN, COOH, halogen, NO₂, CF₃ or SO₃H: R₄ and R₅, which may be the same or different, are H, lower (C₁-C₆) alkyl or substituted lower (C₁-C₆) alkyl in which the substituent may be OH, OR, NHCOR₆, NHCONH₂, NHCO₂R₆, NHCSNH₂, CN, COOH, SO₃H, SO₂NR₆, SO₂R₆ or CO₂R ₆; R₆ is lower (C₁-C₆) alkyl, lower (C₁-C₆) hydroxyalkyl phenyl linked to the nitrogen by an alkylene chain, phenyl or substituted phenyl with the substituent defined as R₁, and R is C₁-C₆ alkyl or C₁-C₆ hydroxyalkyl.

Also included herein are oxidative hair coloring agents of the formula:

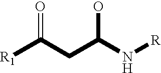

wherein: R₁=substituted or unsubstituted benzene ring, tertiary-butyl, etc.; R=substituted or unsubstituted benzene ring and the formula:

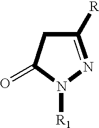

wherein R=aminoalkyl, amidoalkyl, aminobenzene (substituted or unsubstituted), amidobenzene (substituted or unsubstituted), alkyl, substituted or unsubstituted benzene ring ; R₁=substituted or unsubstituted benzene ring.

The primary intermediates can be used herein alone or in combination with other primary intermediates, and one or more can be used in combination with one or more couplers. The choice of primary intermediates and couplers will be determined by the color, shade and intensity of coloration which is desired. There are nineteen preferred primary intermediates and couplers which can be used herein, singly or in combination, to provide dyes having a variety of shades ranging from ash blonde to black; these are: pyrogallol, resorcinol, p-toluenediamine, p-phenylenediamine, o-phenylenediamine, m-phenylenediamine, o-aminophenol, p-aminophenol, 4-amino-2-nitrophenol, nitro-p-phenylenediamine, N-phenyl-p-phenylenediamine, m-aminophenol, 2-amino-3-hydroxypyridine, 1-napthol, N,N bis (2-hydroxyethyl)p-phenylenediamine, 4-amino-2-hydroxytoluene, 1,5-dihydroxynapthalene, 2-methyl resorcinol and 2,4-diaminoanisole. These can be used in the molecular form or in the form of peroxide-compatible salts, as detailed above.

For example low intensity colors such as natural blond to light brown hair shades generally comprise from about 0.001% to about 5%, preferably from about 0.1% to about 2%, more preferably from about 0.2% to about 1% by weight of coloring composition of total oxidative dyeing agents and may be achieved by the combination of primary intermediates such as 1,4-diamino-benzene, 2,5-diamino toluene, 2,5-diamino-anisole, 4-aminophenol, 2,5-diamino-benzyl alcohol and 2-(2′, 5′-diamino)phenyl-ethanol with couplers such as resorcinol, 2-methyl resorcinol or 4-chloro resorcinol.

Similarly combination of the above primary intermediates with couplers, such as, 5-amino-2-methyl phenol and 1,3-diamino-benzene derivatives such as 2,4-diamino-anisole at levels of from about 0.5% to about 1% of total dyeing agents can lead to medium intensity red colors. High intensity colors such as blue to blue-violet hair shades can be produced by the combination of the above primary intermediates with couplers such as 1,3-diamino-benzene or its derivatives such as 2,5-diamino-toluene at levels of from about 1% to about 6% by weight of composition of total dyeing agents. Black hair colors can be obtained by combining the aforementioned primary intermediates with couplers such as 1,3-diaminobenzene or its derivatives

Non-Oxidative and Other Dyes

The hair coloring compositions of the present invention may, in addition to or instead of an oxidative hair coloring agent, include non-oxidative and other dye materials. Optional non-oxidative and other dyes suitable for use in the hair coloring compositions and processes according to the present invention include both semi-permanent, temporary and other dyes. Non-oxidative dyes as defined herein include the so-called ‘direct action dyes’, metallic dyes, metal chelate dyes, fibre reactive dyes and other synthetic and natural dyes. Various types of non-oxidative dyes are detailed in: ‘Chemical and Physical Behaviour of Human Hair’ 3rd Ed. by Clarence Robbins (pp250-259); ‘The Chemistry and Manufacture of Cosmetics’. Volume IV. 2nd Ed. Maison G. De Navarre at chapter 45 by G.S. Kass (pp841-920); ‘cosmetics: Science and Technology’ 2nd Ed., Vol. II Balsam Sagarin, Chapter 23 by F. E. Wall (pp 279-343); ‘The Science of Hair Care’ edited by C. Zviak, Chapter 7 (pp 235-261) and . ‘Hair Dyes’, J. C. Johnson, Noyes Data Corp., Park Ridge, U.S.A. (1973), (pp 3-91 and 113-139).

Direct action dyes which do not require an oxidative effect in order to develop the color, are also designated hair tints and have long been known in the art. They are usually applied to the hair in a base matrix which includes surfactant material. Direct action dyes include nitro dyes such as the derivatives of nitroamino benzene or nitroaminophenol; disperse dyes such as nitroaryl amines, aminoanthraquinones or azo dyes; anthraquinone dyes, naphthoquinone dyes; basic dyes such as Acridine Orange C.I. 46005. In the case of direct dyes, these are preferably used in addition to an oxidative dye and are typically not used on their own in the compositions herein.

Nitrodyes are added to dyeing compositions to enhance the colour of the colorant and to add suitable aesthetic colour to the dye mixture prior to application.

Further examples of direct action dyes include the Arianor dyes basic brown 17, C.I. (color index)—no. 12,251; basic red 76, C.I.—12,245; basic brown 16, C.I.—12,250; basic yellow 57, C.I.—12,719 and basic blue 99, C.I.—56,059 and further direct action dyes such as acid yellow 1, C.I.—10,316 (D&C yellow no. 7); acid yellow 9, C.I.—13,015; basic violet C.I.—45,170; disperse yellow 3, C.I.—11,855; basic yellow 57, C.I.—12,719; disperse yellow 1, C.I.—10,345; basic violet 1, C.I.—42,535, basic violet 3, C.I.—42,555; greenish blue, C.I.—42090 (FD&C Blue no. 1); yellowish red, C.I.—14700 (FD&C red no. 4); yellow, C.I.19140 (FD&C yellow no. 5); yellowish orange, C.I.15985 (FD&C yellow no. 6); bluish green, C.I.42053 (FD&C green no. 3); yellowish red, CI.16035 (FD&C red no. 40); bluish green, C.I.61570 (D&C green no. 3); orange, C.I.45370 (D&C orange no. 5); red, C.I.15850 (D&C red no. 6); bluish red, C.I.15850(D&C red no. 7); slight bluish red, C.I.45380(D&C red no. 22); bluish red, C.I.45410(D&C red no. 28); bluish red, C.I.73360(D&C red no. 30); reddish purple, C.I.17200(D&C red no. 33); dirty blue red, C.I.15880(D&C red no. 34); bright yellow red, C.I.12085(D&C red no. 36); bright orange, C.I.15510(D&C orange no. 4); greenish yellow, C.I.47005(D&C yellow no. 10); bluish green, C.I.59040(D&C green no. 8); bluish violet, C.I.60730(Ext. D&C violet no. 2); greenish yellow, C.I. 10316(Ext. D&C yellow no. 7);

Fibre reactive dyes include the Procion (RTM), Drimarene (RTM), Cibacron (RTM), Levafix (RTM) and Remazol (RTM) dyes available from ICI, Sandoz, Ciba-Geigy, Bayer and Hoechst respectively.

Natural dyes and vegetable dyes as defined herein include henna (Lawsonia alba), camomile (Matricaria chamomila or Anthemis nobilis), indigo, logwood and walnut hull extract.

Temporary hair dyes, or hair coloring rinses, are generally comprised of dye molecules which are too large to diffuse into the hair shaft and which act on the exterior of the hair. They are usually applied via a leave-in procedure in which the dye solution is allowed to dry on the hair surface. As such these dyes are typically less resistant to the effects of washing and cleaning the hair with surface active agents and are washed off of the hair with relative ease. Any temporary hair dye may suitably be used in the compositions of the invention and examples of preferred temporary hair dyes are illustrated below. In the case of temporary dyes, these are preferably used in addition to an oxidative dye and not used on their own in the present compositions.

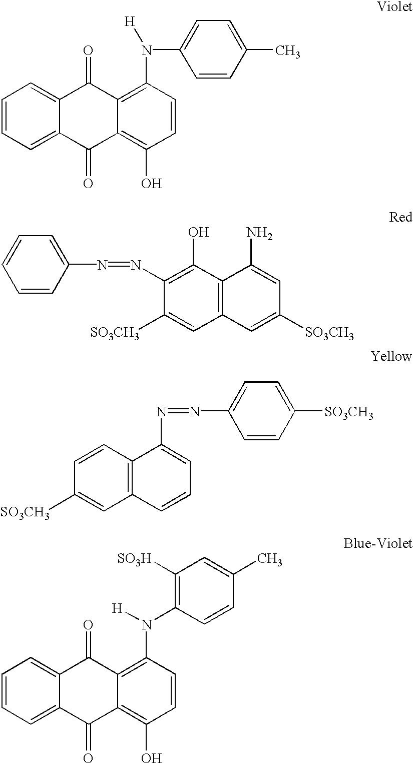

Semi-permanent hair dyes are dyes which are generally smaller in size and effect to temporary hair rinses but are generally larger than permanent (oxidative) dyes. Typically, semi-permanent dyes act in a similar manner to oxidative dyes in that they have the potential to diffuse into the hair shaft. However, semi-permanent dyes are generally smaller in size than the aforementioned conjugated oxidative dye molecules and as such are pre-disposed to gradual diffusion out of the hair again. Simple-hair washing and cleaning action will encourage this process and in general semi-permanent dyes are largely washed out of the hair after about 5 to 8 washes. Any semi-permanent dye system may be suitably used in the compositions of the present invention. Suitable semi-permanent dyes for use in the compositions of the present invention are HC Blue 2, HC Yellow 4, HC Red 3, Disperse Violet 4, Disperse Black 9, HC Blue 7, HC Yellow 2, Disperse Blue 3, Disperse violet 1 and mixtures thereof. Examples of semi-permanent dyes are illustrated below:

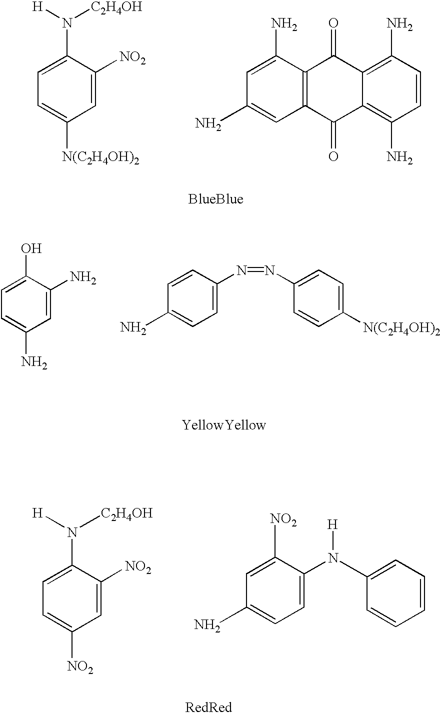

Typical semi-permanent dye systems incorporate mixtures of both large and small color molecules. As the size of the hair is not uniform from root to tip the small molecules will diffuse both at the root and tip, but will not be retained within the tip, while the larger molecules will be generally only be able to diffuse into the ends of the hair. This combination of dye molecule size is used to help give consistent color results from the root to the tip of the hair both during the initial dyeing process and during subsequent washing.

Oxidising Agents

The compositions of the invention preferably also comprise at least one oxidising agent, which may be an inorganic or organic oxidising agent. The oxidising agent is preferably present at a level of from about 0.01% to about 10%, preferably from about 0.01% to about 6%, more preferably from about 1% to about 4% by weight of composition.

Inorganic Oxidising Agents

A preferred oxidising agent for use herein is an inorganic peroxygen oxidising agent. The inorganic peroxygen oxidising agent should be safe and effective for use in the compositions herein. Preferably, the inorganic peroxygen oxidising agents suitable for use herein will be soluble in the compositions according to the present invention when in liquid form or in the form intended to be used. Preferably, inorganic peroxygen oxidising agents suitable for use herein will be water-soluble. Water soluble oxidising agents as defined herein means agents which have a solubility to the extent of about 10 g in 1000 ml of deionised water at 25□ C. (“Chemistry” C. E. Mortimer. 5th Edn. p277).

The inorganic peroxygen oxidising agents useful herein are generally inorganic peroxygen materials capable of yielding peroxide in an aqueous solution. Inorganic peroxygen oxidising agents are well known in the art and include hydrogen peroxide, inorganic alkali metal peroxides such as sodium periodate, sodium perbromate and sodium peroxide, and inorganic perhydrate salt oxidising compounds, such as the alkali metal salts of perborates, percarbonates, perphosphates, persilicates, persulphates and the like. These inorganic perhydrate salts may be incorporated as monohydrates, tetrahydrates etc. Mixtures of two or more of such inorganic peroxygen oxidising agents can be used if desired. While alkali metal bromates and iodates are suitable for use herein the bromates are preferred. Highly preferred for use in the compositions according to the present invention is hydrogen peroxide.

In preferred compositions according to the present invention the inorganic peroxygen oxidising agent is present at a level of from about 0.01% to less than about 6%, preferably from about 0.01% to about 4%, more preferably from about 1% to about 4%, more preferably from about 2% to about 3% by weight of composition.

Preformed Organic Peroxyacid

The compositions according to the present invention may instead or in addition to the inorganic peroxygen oxidising agent(s), comprise one or more preformed organic peroxyacid oxidising agents.

Suitable organic peroxyacid oxidising agents for use in the coloring compositions according to the present invention have the general formula:

R—C(O)OOH

wherein R is selected from saturated or unsaturated, substituted or unsubstituted, straight or branched chain, alkyl, aryl or alkaryl groups with from 1 to 14 carbon atoms.

A class of organic peroxyacid compounds suitable for use herein are the amide substituted compounds of the following general formulae:

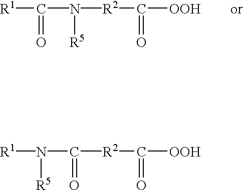

wherein R¹ is, a saturated or unsaturated alkyl or alkaryl group, or an aryl group, having from 1 to 14 carbon atoms, R² is, a saturated or unsaturated alkyl or alkaryl group, or an aryl group, having from 1 to 14 carbon atoms, and R⁵ is H or, a saturated or unsaturated alkyl or alkaryl group, or an aryl group, having from 1 to 10 carbon atoms. Amide substituted organic peroxyacid compounds of this type are described in EP-A-0, 170,386.

Other suitable organic peroxyacid oxidising agents include peracetic, pernanoic, nonylamidoperoxycaproic acid (NAPCA), perbenzoic, m-chloroperbenzoic, di-peroxy-isophthalic, mono-peroxyphthalic, peroxylauric, hexanesulphonyl peroxy propionic, N,N-phthaloylamino peroxycaproic, monoper succinic, nonanoyloxybenzoic, dodecanedioyl-monoperoxybenzoic, nonylamide of peroxyadipic acid, diacyl and tetraacylperoxides, especially diperoxydodecanedioic acid, diperoxytetradecanedioic acid and diperoxyhexadecanedioic acid and derivatives thereof. Mono- and diperazelaic acid, mono- and diperbrassylic acid and N-phthaloylaminoperoxicaproic acid and derivatives thereof are also suitable for use herein.

The preformed organic peroxyacid oxidising agents should be safe and effective for use in the compositions herein. Preferably, the preformed organic peroxyacid oxidising agents suitable for use herein will be soluble in the compositions according to the present invention when in liquid form and in the form intended to be used. Preferably, organic peroxyacid oxidising agents suitable for use herein will be water-soluble. Water-soluble preformed organic peroxyacid oxidising agents as defined herein means agents which have a solubility to the extent of about 10 g in 1000 ml of deionised water at 25□C. (“Chemistry” C. E. Mortimer. 5th Edn. p277).

The preferred peroxyacid materials suitable for use herein are selected from peracetic and pernanoic acids and mixtures thereof.

The preformed organic peroxyacid oxidising agent, where present, is preferably present at a level of from about 0.01% to about 8%, more preferably from about 0.1% to about 6%, most preferably from about 0.2% to about 4%, and especially from about 0.3% to about 3% by weight of the hair coloring composition.

When both an inorganic peroxygen oxidising agent and a preformed organic peroxy acid are present in the compositions herein, the weight ratio of the inorganic peroxygen oxidising agent to the preformed organic peroxy acid is preferably in the range of from about 0.0125:1 to about 500:1, more preferably from about 0.0125:1 to about 50:1.

In addition to the inorganic peroxygen oxidising agents and the preformed organic peroxyacid oxidising agents suitable for use herein, the compositions according to the present invention may optionally comprise additional organic peroxides such as urea peroxide, melamine peroxide and mixtures thereof. The level of organic peroxide, where present, is from about 0.01% to about 3%, preferably from about 0.01% to about 2%, more preferably from about 0.1% to about 1.5% and most preferably from about 0.2% to about 1% by weight of composition.

The coloring compositions of the present invention can be formulated over a wide pH range, eg. from about 2 to about 13, but from the viewpoint of providing compositions with reduced damage and reduced skin staining, the compositions are preferably formulated at low pH, preferably preferably have a pH in the range of from about 2 to about 7, preferably from about 2.5 to about 6, more preferably from about 3 to about 6, even more preferably from about 3.5 to about 5.5, and especially from about 4 to about 5.5. The compositions of the invention may also be formulated at high pH, typically from about 8 to about 12, preferably from about 9 to about 11.

The compositions may contain one or more optional buffering agents and/or hair swelling agents (HSAs). Several different pH modifiers can be used to adjust the pH of the final composition or any constituent part thereof. However, preferred compositions herein are substantially free of additional buffering agents and hair swelling agents, i.e. they comprise less than about 1%, preferably less than about 0.5%, more preferably less than about 0.1% by weight of such agents.

This pH adjustment can be effected by using well known acidifying agents in the field of treating keratinous fibres, and in particular human hair, such as inorganic and organic acids such as hydrochloric acid, tartaric acid, citric acid, succinic acid, phosphoric acid and carboxylic or sulphonic acids such as ascorbic acid, acetic acid, lactic acid, sulphuric acid, formic acid, ammonium sulphate and sodium dihydrogenphosphate/phosphoric acid, disodium hydrogenphosphate/phosphoric acid, potassium chloride/hydrochloric acid, potassium dihydrogen phthalate/hydrochloric acid, sodium citrate/hydrochloric acid, potassium dihydrogen citrate/hydrochloric acid, potassium dihydrogencitrate/citric acid, sodium citrate /citric acid, sodium tartarate/ tartaric acid, sodium lactate/lactic acid, sodium acetate/acetic acid, disodium hydrogenphosphate/citric acid and sodium chloride/glycine/hydrochloric acid, succinic acid and mixtures thereof.

Examples of alkaline buffering agents are ammonium hydroxide, ethylamine, dipropylamine, triethylamine and alkanediamines such as 1,3-diaminopropane, anhydrous alkaline alkanolamines such as, mono or di-ethanolamine, preferably those which are completely substituted on the amine group such as dimethylaminoethanol, polyalkylene polyamines such as diethylenetriamine or a heterocyclic amine such as morpholine as well as the hydroxides of alkali metals, such as sodium and potassium hydroxide, hydroxides of alkali earth metals, such as magnesium and calcium hydroxide, basic amino acids such as L-argenine, lysine, alanine, leucine, iso-leucine, oxylysine and histidine and alkanolamines such as dimethylaminoethanol and aminoalkylpropanediol and mixtures thereof. Also suitable for use herein are compounds other than ammonium carbonate or ammonium carbamate that form HCO₃—by dissociation in water (hereinafter referred to as ‘ion forming compounds’). Examples of suitable ion forming compounds are Na₂CO₃, NaHCO₃, K₂CO₃, NH₄HCO₃, CaCO₃ and Ca(HCO₃) and mixtures thereof.

The hair colouring and conditioning compositions according to the present invention, may, as will be described later herein, may comprise a final composition containing a hair coloring agent and a conditioning agent which have been admixed prior to application to the hair or a may comprise single component system. As such, the compositions according to the present invention may comprise coloring kits of a number of separate components.

In oxidising and coloring kits comprising a portion of inorganic peroxygen oxidising agent, such as hydrogen peroxide, which may be present in either solid or liquid form, a buffering agent solution can be used to stabilise hydrogen peroxide. Since hydrogen peroxide is stable in the pH range from 2 to 4, it is preferable to use a buffering agent having a pH within this range. Dilute acids are suitable hydrogen peroxide buffering agents.

Catalyst

The coloring compositions herein may optionally contain a transition metal containing catalyst for the inorganic peroxygen oxidising agents and the, optional, preformed peroxy acid oxidising agent(s). Suitable catalysts for use herein are disclosed in WO98/27945 which is incorporated herein by reference.

Heavy Metal Ion Sequestrant

The coloring compositions of the invention may contain as an optional component a heavy metal ion sequestrant. By heavy metal ion sequestrant it is meant herein components which act to sequester (chelate or scavenge) heavy metal ions. These components may also have calcium and magnesium chelation capacity, but preferentially they show selectivity to binding heavy metal ions such as iron, manganese and copper. Such sequestering agents are valuable in hair coloring compositions as herein described for the delivery of controlled oxidising action as well as for the provision of good storage stability of the hair coloring products.

Heavy metal ion sequestrants are generally present at a level of from about 0.005% to about 20%, preferably from about 0.01% to about 10%, more preferably from about 0.05% to about 2% by weight of the compositions.

Suitable sequestering agents are disclosed in WO98/27945 which is incorporated herein by reference in its entirety.

Thickeners

The coloring compositions of the present invention may additionally include a thickener at a level of from about 0.05% to about 20%, preferably from about 0.1% to about 10%, more preferably from about 0.5% to about 5% by weight. Thickening agents suitable for use in the compositions herein are selected from oleic acid, cetyl alcohol, oleyl alcohol, sodium chloride, cetearyl alcohol, stearyl alcohol, synthetic thickeners such as Carbopol, Aculyn and Acrosyl and mixtures thereof. Preferred thickeners for use herein are Aculyn 22 (RTM), steareth-20 methacrylate copolymer; Aculyn 44 (RTM) polyurethane resin and Acusol 830 (RTM), acrylates copolymer which are available from Rohm and Haas, Philadelphia, Pa., U.S.A. Additional thickening agents suitable for use herein include sodium alginate or gum arabic, or cellulose derivatives, such as methyl cellulose or the sodium salt of carboxymethylcellulose or acrylic polymers.

Diluent

Water is the preferred diluent for the compositions according to the present invention. However, the compositions according to the present invention may include one or more solvents as additional diluent materials. Generally, solvents suitable for use in the coloring compositions of the present invention are selected to be miscible with water and innocuous to the skin. Solvents suitable for use as additional diluents herein include C₁-C₂₀ mono- or polyhydric alcohols and their ethers, glycerine, with monohydric and dihydric alcohols and their ethers preferred. In these compounds, alcoholic residues containing 2 to 10 carbon atoms are preferred. Thus, a preferred group includes ethanol, isopropanol, n-propanol, butanol, propylene glycol, ethylene glycol monoethyl ether, and mixtures thereof. Water is the preferred principal diluent in the compositions according to the present invention. Principal diluent, as defined herein, means, that the level of water present is higher than the total level of any other diluents.

The diluent is present at a level preferably of from about 5% to about 99.98%, preferably from about 15% to about 99.5%, more preferably at least from about 30% to about 99%, and especially from about 50% to about 98% by weight of the compositions herein.

Enzyme

A further additional material useful in the hair coloring compositions according to the present invention is one or more enzymes. Suitable enzymatic materials include the commercially available lipases, cutinases, amylases, neutral and alkaline proteases, esterases, cellulases, pectinases, lactases and peroxidases conventionally incorporated into detergent compositions. Suitable enzymes are discussed in U.S. Pat. Nos. 3,519,570 and 3,533,139 and in WO98/27945 which is incorporated herein by reference in its entirety.

Surfactant Materials

The compositions of the present invention can additionally contain a surfactant system. Suitable surfactants for inclusion in the compositions of the invention generally have a lipophilic chain length of from about 8 to about 22 carbon atoms and can be selected from anionic, cationic, nonionic, amphoteric, zwitterionic surfactants and mixtures thereof. Suitable surfactants for use herein are disclosed in WO98/27945 which is incorporated herein by reference in its entirety.

Optional Materials

A number of additional optional materials can be added to the coloring compositions herein described each at a level of from about 0.001% to about 5%, preferably from about 0.01% to about 3%, more preferably from about 0.05% to about 2% by weight of composition. Such materials include proteins and polypeptides and derivatives thereof; water-soluble or solubilizable preservatives such as DMDM Hydantoin, Germall 115, methyl, ethyl, propyl and butyl esters of hydroxybenzoic acid, EDTA, Euxyl (RTM) K400, natural preservatives such as benzyl alcohol, potassium sorbate and bisabalol, benzoic acid, sodium benzoate and 2-phenoxyethanol; antioxidants such as sodium sulphite, hydroquinone, sodium bisulphite, sodium metabisulphite and thyoglycolic acid, sodium dithionite, erythrobic acid and other mercaptans; dye removers such as oxalic acid, sulphated castor oil, salicylic acid and sodium thiosulphate; H₂O₂ stabilisers such as tin compounds such as sodium stannate, stannic hydroxide and stannous octoate, acetanilide, phenacetin colloidal silica such as magnesium silicate, oxyquinoline sulphate, sodium phosphate, and tetrasodium pyrophosphate; and □-hydroxybenzoates; moisturising agents such as hyaluronic acid, chitin , and starch-grafted sodium polyacrylates such as Sanwet (RTM) IM-1000, IM-1500 and IM-2500 available from Celanese Superabsorbent Materials, Portsmith, Va., U.S.A. and described in U.S. Pat. No. 4,076,663 as well as methyl cellulose, starch, higher fatty alcohols, paraffin oils, fatty acids and the like; solvents ; anti-bacterial agents such as Oxeco (phenoxy isopropanol); low temperature phase modifiers such as ammonium ion sources (e.g. NH₄ Cl); viscosity control agents such as magnesium sulfate and other electrolytes; quaternary amine compounds such as distearyl-, dilauryl-, di-hydrogenated beef tallow-, dimethyl ammonium chloride, dicetyldiethyl ammoniumethylsulphate, ditallowdimethyl ammonium methylsulphate, disoya dimethyl ammonium chloride and dicoco dimethyl ammonium chloride; hair conditioning agents such as silicones, higher alcohols, cationic polymers and the like; enzyme stabilisers such as water soluble sources of calcium or borate species; colouring agents; TiO₂ and TiO₂-coated mica; perfumes and perfume solubilizers; and zeolites such as Valfour BV400 and derivatives thereof and Ca²⁺/Mg²⁺ sequestrants such as polycarboxylates, amino polycarboxylates, polyphosphonates, amino polyphosphonates etc. and water softening agents such as sodium citrate.

Combing Technical Test Method

As mentioned above, the hair colouring and conditioning compositions of the present invention must provide an Average Combing Index Value on wet hair after 6 cycles of 1.2 or greater, preferably of 1.5 or greater, more preferably 1.8 or greater, most preferably 2.0 or greater, as measured by the Combing Technical Test Method described below.

It is also preferable that the hair colouring and conditioning compositions of the present invention provide a Combing Index Value on wet hair after 1 cycle of greater than 1.1 and a Combing Index Value on wet hair after 6 cycles of greater than 1.1.

The hair switches used in this test method are medium brown virgin hair, weighing 6 grams and being 10 inches in length. Five of these hair switches are treated with the hair colouring and conditioning compositions of the present invention as follows.

Treatment of Switches with Colouring and Conditioning Compositions

Colouring and conditioning compositions to be tested are prepared just before the product application, (for example, by mixing a first component containing oxidative hair colouring agents with a second component containing hydrogen peroxide). The switch is hung above a sink and wetted for 30 seconds with water at 100□F. and 1.5 gpm pressure and 8-15grains water hardness where in the test method 1 grain is equivalent to 17.1 ppm of calcium and/or magnesium ions. (The same water hardness is used throughout the experiment). Excess water is squeezed out of the switch. 1.2grams of the colouring composition to be tested is applied on the top end of the switch and spread evenly down to the bottom end, then milked into the switch for 30 seconds and left on the switch for a further 4.5 minutes (5 minutes total application time). The switch is then rinsed with water at 100□F. for 30 seconds. Excess water is squeezed out of the switch. The above treatment constitutes 1 cycles of application. This cycle is repeated up to 6 times, combing measurements are taken after each cycle. As used herein the term “Combing Index Value” is the Combing Index Value (“CIV”) measured after a specified number of cycles. As used herein the term “Average Combing Index Value” is calculated as follows:

Average Combing Index Value=(CIV after 1 cycle+CIV after 2 cycles+CIV after 3 cycles+CIV after 4 cycles+CIV after 5 cycles+CIV after 6 cycles )/6

Combing Measurements

Equipment Needed:

Instron 5543 tensile tester with IBM compatible computer and Merlin Profiler software

Instron load cell, SN maximum capacity with a cross-head adapter

String with small clip

Special comb holder for combing measurements

295 Hercules Saw Cut 2 Combs, combining “wide teeth” part (4 teeth/cm, 9 cm length) and “narrow teeth” part (10 teeth/cm, 8 cm length)

For each test, 5 hair switches per treatment are used, 5 individual measurements per switch are taken. The switches are treated with the colouring compositions to be tested as described above. The wet switches are measured as soon as the treatment cycle is completed.

The load cell is assembled on the cross-head of the Instron tester. Any load cells are removed from the base of the tester. The string is assembled with the clip on the load cell. The comb holder is secured on the base of the Instron tester with screws. The Instron tester is switched on and the transducer is allowed to equilibrate. The computer is switched on and the Merlin Profiler software is downloaded. [Merlin Profiler software is commercially available from the Instron Corporation]. The conditions outlined in the test profile are as follows:

Extension speed: 15 mm/sec

Extension length: 240 mm

Reading time interval : 0.5 sec

The cross-head of the Instron tester is set into the starting position. This position should ensure the distance from the comb to the top of the hair switch is equal to 20 mm. Each hair switch is taken from the rack and combed 1 time with the big teeth and 1 time with the narrow teeth (combing is repeated if required, until there is no tangle left). The new comb is placed into the holder, such that the narrow teeth part is available for combing. The hair switch is secured in the clip, and the load cell is balanced. The switch is then pressed through the narrow teeth of the comb and secured in position by the deflector. The test method is commenced by activating the moving cross-head.

The test must include one control, which is non-treated hair switches wetted with water for 30 seconds (under conditions used with the colouring compositions).

Once all the switches are analysed, the combing work is then calculated for all switches using the following equation (Eq 1.):

$$Work ( mJ ) = {\int }_{x = 0}^{x = 240} F ⅆ x . \qquad \text{(Eq 1.)}$$

Where: F is the load in the load cell when the switch is pulled over a distance x mm through the comb.

The combing work for each hair switch is averaged. The averages of 5 switches is used to produce an average reading per treatment (Work[treatment]). These readings are then analysed for significant differences between treatments using standard statistical methods.

The actual combing work is converted into index readings, based on the results obtained for the internal control (Eq 2.)

$$Combing Index Value = \frac{Work [ control ]}{Work [ treatment ]} \qquad ( Eq 2. )$$

Results

The test method above was carried out on several hair colouring and conditioning products according to the invention and several commercially available hair care products. The Combing Index Values obtained were as follows:

| | | | | | | | Average |
|---|---|---|---|---|---|---|---|
| Treatment Product | 1 cycle | 2 cycles | 3 cycles | 4 cycles | 5 cycles | 6 cycles | CIV |
| Example A¹ | 1.6 | 1.7 | 2.0 | 2.2 | 2.1 | 2.1 | 1.95 |
| Example B² | 1.1 | 0.7 | 0.8 | 0.9 | 0.8 | 0.9 | 0.87 |
| Example C³ | 0.9 | 1.1 | 1.1 | 0.9 | 0.9 | 0.8 | 0.95 |
| Example D⁴ | 1.1 | 1.1 | 1.2 | 0.9 | 0.9 | 0.7 | 0.98 |
| Example E⁵ | 1.1 | 0.8 | 0.8 | 0.8 | 0.8 | 0.7 | 0.83 |
| Example F⁶ | 1.6 | 1.7 | 1.8 | 2.1 | 2.0 | 1.9 | 1.85 |
| Example G⁷ | 1.1 | 1.1 | 1.4 | 1.2 | 1.3 | 1.1 | 1.2 |

1. Example A is a hair colouring and conditioning composition according to the present invention prepared by mixing the composition Example XII below with commercially available Pantene Pro-V Vitalising Conditioner- Balanced for Normal Hair to which a solution of Hydrogen Peroxide has been added, such that the level of hydrogen peroxide in the conditioner composition is 6% by weight of the conditioner composition. Example XII and the hydrogen peroxide containing conditioner composition are mixed in a 1:1 ratio.

2. Example B is a commercially available hair colouring composition which lies outside the scope of the present invention- L'Oreal Castings Light Brown No. 33 (excluding in-box conditioner)—Batch LU211

3. Example C is a commercially available hair colouring composition which lies outside the scope of the present invention- Goldwell Colourance pH 6.8 system No. 5N—Batch D1458P

4. Example D is a commercially available hair colouring composition which lies outside the scope of the present invention—Kao Corp Lavenus Neo Yellow Nuance—Batch 1799

5. Example E is a commercially available hair colouring composition which lies outside the scope of the present invention—L'Oreal Excellence No. 5 (excluding in-box conditioner)—Batch PU344

6. Example F is a hair colouring and conditioning composition according to the present invention prepared by mixing the composition Example XII below with commercially available Pantene Pro-V Vitalising Conditioner—Moisturising for Dry or Damaged Hair to which a solution of Hydrogen Peroxide has been added, such that the level of hydrogen peroxide in the conditioner composition is 6% by weight of the conditioner composition. Example XII and the hydrogen peroxide containing conditioner composition are mixed in a 1:1 ratio.

7. Example G is the composition of Example XII below which lies outside the scope of the present invention.

I Assessment of Initial Color and Color Change

It is preferred that the hair colouring and conditioning compositions of the invention provide a Delta E value of greater than about 8, preferably greater than about 10, more preferably greater than about 12, most preferably greater than about 15, and especially greater than about 20, as measured by the test method below.

Colouring Technical Test Method

Assessment of Color Change

The equipment used to measure both the initial color and color change on substrates (hair/skin) dyed with the colouring and conditioning compositions of the present invention is a Hunter LabScan spectrophotometer. The value used to express the degree of color change on any particular substrate is Delta E (^{□}E). Delta E, as defined herein, is represented by a factual sum of L, a, and b values such that:

^{Δ}E=(^{Δ}L²+^{Δ}a²+^{Δ}b²)½

and L, a, b represent values from the CIELab colour space as defined by Commission Internationale de L'Eclairage (CIE). According to this definition, L is a measure of lightness and darkness, wherein L=100 is equivalent to white, and L=0 is equivalent to black. Further, ‘a’ is a measure of the red and green quotients (color hues) such that positive equates to red and negative equates to green, and ‘b’ is a measure of the yellow and blue quotients (color hues) such that positive equates to yellow and negative equates to blue.

Hunter LabScan Colorimeter is a full scanning spectrocolorimeter with a wavelength of from 400-700 nanometers which records the color of test hair switches (tresses) in terms of ‘L’, ‘a’ and ‘b’ values. The machine is set to: mode—0/45; port size—½ inch; view size—½ inch; light—D65; field of view—10□; UV lamp/filter—none. The hair is placed in a sample holder designed to hold the hair in a uniform orientation during measurement. Equivalent colorimeters can be used, but it must be ensured that the hair does not move during measurement. The hair must be spread to cover the port during color measurement. Dots are placed on the switch holder to guide the positioning of the holder at the port. The dots are lined up with a mark on the port and readings are taken at each spot.

Eight measurements are run per switch, 4 on each side.

Standard Hair Switch

The compositions according to the present invention can be used to color hair of all colors, types and condition. For the purposes of illustration, grey hair switches are used. These standard hair switches can be measured in terms of their approximate L, a, b values.

| | L | a | b |
|---|---|---|---|
| Grey hair switch | 75 | 4 | 27 |

Hair Switch Colouring Method—Single Application

To color hair, a 4 gram switch of about 8 inch long hair is hung over a suitable container and wetted for 30 seconds. The test colouring product is then prepared (i.e., where applicable the separate bottle components are mixed together) and about 5.6 grams of product is applied directly to the test hair switch. The colorant is massaged through the hair switch for up to about 1 minute and then left on the hair switch for about 20 minutes. After rinsing with running water for about 2 minutes the colored hair switch is dried using a hair drier. The color development of the colored and dried test hair switch can then be assessed using the Hunter LabScan spectrophotometer.

Hair Switch Colouring Method—Multiple Applications

To colour hair, a 4 gram switch of about 8 inch long hair is hung over a suitable container and wetted for 30 seconds. The test colouring product is then prepared (i.e., where applicable the separate bottle components are mixed together) and about 0.4 grams of product is applied directly to the test hair switch. The colorant is massaged through the hair switch for up to about 30 seconds and then left on the hair switch for about 1 minute. After rinsing with running water for about 2 minutes the colored hair switch is dried using a hair drier. The color development of the colored and dried test hair switch can then be assessed using the Hunter LabScan spectrophotometer. The above protocol is repeated for further 13 cycles. The final colour development of the dried test hair switch is assessed using the Hunter LabScan spectrophotometer and represents 14 applications of the colouring and conditioning composition. The present invention is represented by the following non-limiting examples. In the examples, all concentrations are on a 100% active basis and all percentages are by weight unless otherwise stated and the abbreviations have the following designations.

### EXAMPLES I-XV

Examples I-XV are compositions containing oxidative hair colouring agents. Any of these compositions can be mixed with the composition of Example XVI in a 1:1 ratio in order to provide a hair colouring and conditioning composition according to the present invention.

| | | | | | | | | |
|---|---|---|---|---|---|---|---|---|
| Ingredient | I | II | III | IV | V | VI | VII | VIII |

Dye Premix:

| | | | | | | | | |
|---|---|---|---|---|---|---|---|---|
| Demineralized Water | 45.9 | 45.77 | 45.83 | 44.6 | 44.56 | 43.9 | 44.91 | 43.31 |
| Citric Acid | 1.7 | 1.7 | 1.7 | 1.7 | 1.7 | 1.7 | 1.7 | 1.7 |
| Tetrasodium EDTA | 0.19 | 0.19 | 0.19 | 0.19 | 0.19 | 0.19 | 0.19 | 0.19 |
| Sodium Sulphite | 0.2 | 0.2 | 0.2 | 0.2 | 0.2 | 0.2 | 0.2 | 0.2 |
| pPD¹ | 0 | 0.1 | 0 | 0.24 | 0 | 0.4 | 0.332 | 0.2 |
| mPD sulphate² | 0 | 0.001 | 0 | 0 | 0 | 0 | 0 | 0 |
| pAP³ | 0 | 0.012 | 0 | 0.27 | 0.75 | 0.9 | 0.332 | 1.498 |
| 2A3HP⁴ | 0 | 0 | 0 | 0.6 | 0.6 | 0.6 | 0 | 0.6 |
| mAP⁵ | 0 | 0.012 | 0 | 0 | 0 | 0.1 | 0 | 0 |
| resorcinol | 0 | 0 | 0 | 0 | 0 | 0 | 0 | 0 |
| 4A2HT⁶ | 0 | 0.006 | 0.04 | 0 | 0 | 0 | 0 | 0 |
| o-AP⁷ | 0 | 0 | 0.04 | 0 | 0 | 0 | 0.332 | 0 |
| 32% NaOH | 0 | 0 | 0 | 0 | 0 | 0 | 0 | 0 |
| Ascorbic Acid | 0 | 0 | 0 | 0.2 | 0 | 0 | 0 | 0.3 |
| Dye Wash Water: | | | | | | | | |
| Water | 8 | 8 | 8 | 8 | 8 | 8 | 8 | 8 |
| Emulsion Base: | | | | | | | | |
| Water | 29.77 | 29.77 | 29.77 | 29.77 | 29.77 | 29.77 | 29.77 | 29.77 |
| Ceteareth-25 | 1.5 | 1.5 | 1.5 | 1.5 | 1.5 | 1.5 | 1.5 | 1.5 |
| Cetyl Alcohol | 2.25 | 2.25 | 2.25 | 2.25 | 2.25 | 2.25 | 2.25 | 2.25 |
| Stearyl Alcohol | 2.25 | 2.25 | 2.25 | 2.25 | 2.25 | 2.25 | 2.25 | 2.25 |
| Sodium Benzoate | 0.09 | 0.09 | 0.09 | 0.09 | 0.09 | 0.09 | 0.09 | 0.09 |
| Phenoxyethanol | 0.108 | 0.108 | 0.108 | 0.108 | 0.108 | 0.108 | 0.108 | 0.108 |
| Tetrasodium EDTA | 0.036 | 0.036 | 0.036 | 0.036 | 0.036 | 0.036 | 0.036 | 0.036 |
| pH adjusters: | | | | | | | | |
| Water | to 100 | to 100 | to 100 | to 100 | to 100 | to 100 | to 100 | to 100 |
| 50% citric acid | 4 | 0 | 0 | 0 | 0 | 0 | 0 | 0 |
| 32% NaOH | 0 | 1.8 | 2 | 1.5 | 0.7 | 0.5 | 1.75 | 0.467 |

| Ingredient | IX | X | XI | XII | XIII | XIV | XV |
|---|---|---|---|---|---|---|---|
| Dye Premix: | | | | | | | |
| Demineralized Water | 43.31 | 42.21 | 43.81 | 42.51 | 41.38 | 43.84 | 38.45 |
| Citric Acid | 1.7 | 1.7 | 1.7 | 1.7 | 1.7 | 1.7 | 1.7 |
| Tetrasodium EDTA | 0.19 | 0.19 | 0.19 | 0.19 | 0.19 | 0.19 | 0.19 |
| Sodium Sulphite | 0.2 | 0.2 | 0.2 | 0.2 | 0.2 | 0.2 | 0.2 |
| pPD¹ | 0.8 | 0.6 | 0 | 0.8 | 1.9 | 0.95 | 2 |
| mPD sulphate² | 0 | 0 | 0 | 0 | 0 | 0 | 0.4 |
| pAP³ | 0.6 | 1.498 | 1.498 | 1.498 | 0.54 | 0.27 | 0 |
| 2A3HP⁴ | 0 | 0 | 0 | 0 | 0.44 | 0.22 | 0 |
| mAP⁵ | 0 | 0.6 | 0.6 | 0.5 | 0.048 | 0.024 | 0 |
| resorcinol | 0 | 0 | 0 | 0 | 0 | 0 | 0.45 |
| 4A2HT⁶ | 0 | 0 | 0 | 0 | 0 | 0 | 0 |
| o-AP⁷ | 0 | 0 | 0 | 0 | 0 | 0 | 0 |
| 32% NaOH | 0 | 0 | 0 | 0 | 0 | 0 | 3.003 |
| Ascorbic Acid | 0.6 | 1 | 0 | 0.6 | 1.6 | 0.6 | 1.6 |
| Dye Wash Water: | | | | | | | |
| Water | 8 | 8 | 8 | 8 | 8 | 8 | 8 |
| Emulsion Base: | | | | | | | |
| Water | 29.77 | 29.77 | 29.77 | 29.77 | 29.77 | 29.77 | 29.77 |
| Ceteareth-25 | 1.5 | 1.5 | 1.5 | 1.5 | 1.5 | 1.5 | 1.5 |
| Cetyl Alcohol | 2.25 | 2.25 | 2.25 | 2.25 | 2.25 | 2.25 | 2.25 |
| Stearyl Alcohol | 2.25 | 2.25 | 2.25 | 2.25 | 2.25 | 2.25 | 2.25 |
| Sodium Benzoate | 0.09 | 0.09 | 0.09 | 0.09 | 0.09 | 0.09 | 0.09 |
| Phenoxyethanol | 0.108 | 0.108 | 0.108 | 0.108 | 0.108 | 0.108 | 0.108 |
| Tetrasodium EDTA | 0.036 | 0.036 | 0.036 | 0.036 | 0.036 | 0.036 | 0.036 |
| pH adjusters: | | | | | | | |
| Water | to 100 | to 100 | to 100 | to 100 | to 100 | to 100 | to 100 |
| 50% citric acid | 0 | 0 | 0 | 0 | 0 | 0 | 0 |
| 32% NaOH | 1.8 | 1.5 | 0.5 | 1.6 | 3.7 | 1.1 | 2 |

¹paraphenylenediamine 2HCl
²metaphenylenediamine sulphate
³para-aminophenol
⁴2-amino-3-hydroxypyridine
⁵meta-aminophenol
⁶4-amino-2-hydroxytoluene
⁷ortho-aminophenol

In the examples above, water is used as the diluent. However in variations hereof water can be replaced, in part, by from about 0.5% to about 50% by weight of the total water content of the examples by diluents such as lower alcohols, e.g., ethylene glycol, ethylene glycol monoethyl ether, diethylene glycol, diethylene glycol monoethyl ether, propylene glycol, 1,3-propanediol, ethanol, isopropyl alcohol, glycerine, butoxyethanol, ethoxydiglycol, hexylene glycol, polyglyceryl-2-oleyl ether and mixtures thereof.

The compositions of Examples of I-XV are prepared as follows:

The dye premix is prepared by adding to a vessel with stirring the deionised water, sodium sulphite, tetrasodium EDTA, ammonium actetae and dipropylene glycol. The dye materials are then added to the vessel and mixed until dissolved, with heating if necessary. When fully dissolved, the solution is cooled to room temperature, followed by addition of ascorbic acid.

The pH premix is prepared by mixing together in a vessel the sodium hydroxide and deionised water.

The emulsion base is prepared by adding to a vessel the deionized water and commencing agitation with heating to 80□C. Then the tetrasodium EDTA is added followed by the sodium benzoate and these are dissolved. This is followed by addition of ceteareth. The homogenizer is then turned on and the cetyl alcohol and stearyl alcohol are added keeping the temperature above 80□C. When homogenized, the mixture is cooled to 60□C. when phenoxytol is added. At 40□C. the homogenizer and mixer is stopped and the mixture is left to thicken for 60 minutes.

The final dye base is made by adding the emulsion base to a vessel, followed by addition of the dye premix solution, and the resulting mixture is mixed until homogeneous. Ammonium hydroxide is added followed by mixing and addition of the pH premix. Water is added to 100% and mixed until homogeneous.

Any of Examples I-XV above is mixed in a 1:1 ratio with the conditioning composition below in Example XVI or Example XVII.

Example XVI is commercially available Pantene Pro-V Vitalising Conditioner—Balanced for normal hair, to which a solution of hydrogen peroxide has been added such that Example XVI contains 6% by weight of hydrogen peroxide. Example XVII is commercially available Pantene Pro-V Vitalising Conditioner—Moisturising for Dry or Damaged Hair, to which a solution of hydrogen peroxide has been added such that Example XVII contains 6% by weight of hydrogen peroxide.

The hair colouring and conditioning compositions obtained by mixing any of Examples I-XV with either Example XVI or Example XVII in a 1:1 ratio, provide a complete range of colours, i.e. from blondes to blacks, while providing superior hair conditioning. The compositions of the examples also provide excellent grey coverage, and excellent coverage of the roots, when used as a conventional hair conditioning composition about 4 times a week.

Method of Use

It is important that dyeing compositions be in a form which is easy and convenient to prepare and use by the consumer, since the oxidising agent must remain in contact with the hair for a certain period of time and not run or drip off of the hair, possibly causing eye or skin irritation.

To address the above, the coloring compositions of the present invention can be provided in both a single pack or in kit form as separately packaged components to maintain stability, and, if so desired, either mixed by the user immediately prior to application to the hair, or mixed and stored for future use, or mixed and partly used and the remainder stored for future use.

As hereinbefore described, the compositions, according to the present invention, may be used by the consumer as a single component package. Such a single pack would comprise a single composition containing the hair coloring agent and the hair conditioning agent. The composition would be applied directly to the hair by the consumer without the need for any pretreatments or mixing thereby providing a simple, fast, easy to use, ‘no-mess’ hair colouring and conditioning system. A further advantage of such a single component system is that it could be stored and re-used i.e., a single package could contain enough colouring composition for several applications over time. In addition, the compositions may be dispensed from a pack having dual chambers, each chamber comprising a single component, wherein the single components are intended to be mixed, either within the pack or outside the pack to form a homogeneous composition, which can be applied to the hair.

Thus, according to a further aspect of the present invention, there is provided a method for colouring and conditioning the hair wherein a hair colouring and conditioning composition is applied directly to wet hair and subsequently rinsed off wherein the hair coloring and conditioning composition comprises:

(b) a conditioning agent; and

(c) a hair coloring agent;

wherein the composition has an Average Combing Index Value of greater than 1.2 as measured by the Combing Technical Test Method described hereinabove.

The colouring and conditioning compositions herein are preferably applied to the hair like a conventional conditioning rinse composition and rinsed from the hair after use. The colouring and conditioning compositions are thereby easy and convenient to use in the shower. The colouring and conditioning compositions are preferably applied to the hair for periods of from 0.5 minutes to 5 minutes depending upon the degree of coloring required. A preferred time is between 0.5 minutes and 3 minutes. The coloring compositions according to the present invention is preferably applied to wet hair after the hair has been shampooed.

Kits

According to the present invention there is also provided a hair coloring kit wherein the kit comprises a package which contains two or more separate components and preferably also instructions directing the consumer how to use the kit for colouring the hair. In preferred embodiments the kit components are admixed to form a colouring and conditioning composition prior to application to the hair. In alternative embodiments, the individual components can be applied separately to the hair in a sequential manner.

A preferred kit herein comprises a package comprising at least two components, a first component containing a combination of an oxidative and/or non-oxidative hair coloring agent and a conditioning agent and a second component comprising an oxidising agent and a conditioning agent. The components can either be mixed by the user immediately prior to application to the hair or can be applied separately. The components can also be mixed and the resulting composition can be stored for future use or part of the resulting composition can be used and the rest of the composition stored for future use. In one embodiment of the present invention the oxidising component comprises a stabilised cream comprising an inorganic peroxygen oxidising agent, most generally hydrogen peroxide in an amount such that the final concentration of the coloring composition for use on the hair is from about 0.05% to about 6% by weight and additional agents as herein before described.

In kits comprising two or more components, these components may be packaged separately or may be packaged in a single package having several chambers, one chamber for each separate component.

A preferred pack herein is a pack having two chambers, one chamber comprising a first component comprising a hair colouring agent and a second chamber comprising a second component comprising an oxidising agent. Either or both of the second components may contain a conditioning agent, provided at least one of the components contains a conditioning agent. The two components can be mixed either outside the pack or within the pack. It is important that the two components are mixed sufficiently in order to provide a homogeneous hair colouring and conditioning composition for application to the hair. Such a pack provides a product which is easy and convenient to use in the shower.

An example of a kit according to the present invention is a package having two chambers, one chamber containing the composition of Example XVI or Example XVII and the other chamber containing any one of the compositions of Examples I-XV. The pack has a means of mixing a defined level each of the two compositions within the pack and a means to dispense the resulting hair colouring and conditioning composition. The kit also contains instructions instructing consumers that they should use in the shower on wet hair, after shampooing, in place of their normal conditioning composition, preferably from once and 7 times a week, preferably between about twice and 4 times a week.

What is claimed is:

1. A hair colouring and conditioning composition comprising:
(a) at least one conditioning agent; and
(b) at least one hair colouring agent;
wherein the composition has an Average Combing Index Value on wet hair of greater than 1.2 as measured by the Combing Technical Test Method as defined herein.

2. A composition according to claim 1 wherein the composition has an Average Combing Index Value of greater than 1.5.

3. A composition according to claim 1 wherein the composition has an Average Combing Index Value of greater than 1.8.

4. A composition according to claim 1 wherein the conditioning agent is selected from cationic surfactant, cationic conditioning polymer, silicone conditioning agent, fatty alcohol, fatty acid, and mixtures thereof.

5. A composition according to claim 1 wherein the conditioning agent is selected from cationic surfactants, cationic conditioning polymers, insoluble silicone conditioning agents, non-volatile hydrocarbon esters, and fatty alcohols and mixtures thereof.

6. A composition according to claim 1 wherein the conditioning agent is a mixture of cationic conditioning polymer, silicone conditioning agent and fatty alcohol.

7. A composition according to claim 1 comprising from about 0.1% to about 25% by weight of conditioning agent.

8. A composition according to claim 1 wherein the hair colouring agent is an oxidative hair colouring agent.

9. A composition according to claim 1 additionally comprising from about 0.01% to less than about 6% by weight of composition of oxidising agent.

10. A composition according to claim 9 wherein the oxidising agent is an inorganic peroxygen oxidising agent.

11. A composition according to claim 9 wherein the oxidising agent is hydrogen peroxide.

12. A composition according to claim 1 wherein the pH of the composition is in the range of from about 2 to about 13.

13. A composition according to claim 1 wherein the total level of hair coloring agent is from about 0.001% to about 5% by weight.

14. A composition according to claim 1 wherein the composition has a Combing Index Value after 1 cycle of greater than 1.1 and a Combing Value after 6 cycles of greater than 1.1.

15. A composition for simultaneously colouring and conditioning the hair comprising:
(a) an oxidative hair colouring agent
(b) an oxidising agent
(c) a conditioning component
wherein the conditioning component comprises a material selected from the group consisting of:
from about 0.1% to about 10% by weight of composition of insoluble silicone conditioning agent,
from about 0.1% to about 10% by weight of a cationic conditioning agent, from about 0.1% to about 10% by weight of a straight chain fatty alcohol, and mixtures thereof.

16. Use of a composition according to claim 1 for colouring and conditioning human or animal hair.

17. A method for colouring and conditioning human or animal hair comprising applying to the hair a hair colouring and conditioning composition according to claim 1 and subsequently rinsing it off.

18. A hair colouring and conditioning kit comprising at least two components, a first component comprising an oxidising agent and optionally a conditioning agent and a second component comprising an oxidative hair coloring agent and optionally a conditioning agent, provided at least one of the components comprises a conditioning agent, wherein the two components are mixed together immediately before application to the hair to form the hair colouring and conditioning composition of claim 1.

19. A packaged hair colouring and conditioning product comprising a package comprising at least two chambers, a first chamber containing a first component comprising an oxidative hair colouring agent and a second chamber containing a second composition comprising an oxidising agent, wherein the first composition and the second composition are mixed either inside or outside the package before application to the hair, to provide a hair colouring and conditioning composition, wherein the hair colouring and conditioning composition comprises at least one conditioning agent and at least one hair colouring agent and wherein the hair colouring and conditioning composition provides an Average Combing Index Value of greater than 1.2 as measured by the Combing Technical Test Method as defined herein.

20. A kit according to claim 18 additionally comprising an initial colouring composition suitable for providing an initial colour to hair, wherein the colour provided by the hair colour altering composition (a) is substantially the same shade as the initial colouring composition.
